(19) United States
(12) Patent Application Publication
(10) Pub. No.: US 2006/0074047 A1
(43) Pub. Date: Apr. 6, 2006

(54) Method of treating inflammation with inhibitors of sialyl transferases

(76) Inventors: Alan S. Cross, Chevy Chase, MD (US); Nicholas M. Stamatos, Baltimore, MD (US); Simeon E. Goldblum, Baltimore, MD (US)

Correspondence Address: Benjamin Aaron Adler;ADLER & ASSOCIATES, 8011 Candle Lane, Houston, TX, 77071, US

(21) Appl. No.: 11/244,551

(22) Filed: Oct. 6, 2005

Related U.S. Application Data
(60) Provisional application No. 60/615,899, filed on Oct. 6, 2004.

(51) Int. Cl. A61K 31/7072

(52) U.S. Cl. 514/49

(57) ABSTRACT

Provided herein are a methods of reducing a level of activity of a sialyl transferase enzyme, a sialidase enzyme or a combination thereof using inhibitors of these enzymes, for example specific inhibitory compound or antibodies directed against the enzymes. The methods are effective to treat an inflammatory disease or disorder or a primary or metastatic cancer. Also provided is a method for screening potential inhibitors of sialyl transferase enzymes.

### CROSS-REFERENCE TO RELATED APPLICATION

This non-provisional application claims benefit of provisional U.S. Ser. No. 60/615,899, filed Oct. 6, 2004, now abandoned.

### FEDERAL FUNDING LEGEND

The present invention was generated at least in part with funds from the National Institutes of Health, NIH Grant No. A142818. The United States Government may have certain rights in the invention.

### BACKGROUND OF THE INVENTION

1. Field of the Invention

The present invention relates generally to the fields of enzymology and diseases and disorders involving inflammation or a cancer. More specifically, the present invention relates to inhibitors of sialyl transferases and their uses as agents against inflammatory diseases or disorders or cancer.

2. Description of the Related Art

Inflammation is often accompanied by an influx of polymorphonuclear leukocytes (PMNs) from the circulation to sites of inflammation. This requires that polymorphonuclear leukocytes migrate across the endothelial cells that line the blood vessels in a process called diapedesis (1). Polymorphonuclear leukocytes adhere endothelial cells that overlie sites of inflamed tissue, and then migrate between the endothelial cells and continue to the nidus of infection. Part of this process includes the activity of sialyltransferases (ST) on the polymorphonuclear leukocytes, and consequently, inhibition of sialyltransferases activity diminishes the migration and therefore the recruitment of polymorphonuclear leukocytes to the inflammation.

Sialic acids, or N-acetylneuraminic acids, are a family of amino sugars that are coupled to the outermost ends of glyconjugates on the surface of eukaryotic cells and impart a negative charge to both the molecule and the cell. Sialic acid residues on the cell surface reduce cell-to-cell adhesion, prevent the deposition of complement on the cell surface, permit evasion of immune recognition and may prevent the binding of ligands to their cell surface receptors (2). Sialyltransferases are a family of enzymes that catalyze the transfer of N-acetyineuraminic acid from CMP-β-N-acetylneuraminic acid onto carbohydrate groups of glycolipids and glycoproteins, i.e., glycoconjugates, usually at the terminal position. More than 10 distinct eukaryotic sialyltransferases have been identified which differ in substrate specificity for acceptor sugars, e.g. galactose, N-acetylgalactosamine or sialic acid, as well as the type of glycosidic linkages formed, e.g. α2,3, α2,6, α2,8.

Enzymatic modification of the sialylation status of these cell surface glycoconjugates may provide an additional layer of regulation for polymorphonuclear leukocyte interactions with other cells or informational molecules. Microbial neuraminidases and cellular sialidases, by removing sialyl residues from these surface glycoconjugates, can rapidly modify their affinity for binding partners (3). Upon activation, human polymorphonuclear leukocytes mobilize endogenous sialidase activity from intracellular compartments to the plasma membrane where it cleaves sialyl residues from cell surface glycoconjugates. Such desialylation results in marked polymorphonuclear leukocyte functional changes, including increased adhesiveness to and migration across the endothelium.

Sialyltransferases are preferentially localized to the Golgi apparatus within the cell; however, extensive work by Shur and colleagues have established that glycosyltransferases may be expressed on the cell surface where they function as cell adhesion molecules, signal transducing receptors for extracellular oligosaccharide ligands, and regulators of cell growth. Ecto-STs have been described in platelets, lymphoblastoid cells, B lymphocytes, neuronal cells and more recently, on the surface of early-activated CD8 T cells. Since there appears to be a connection between the Golgi and the cell surface, Golgi-associated ST might be exported to the plasma membrane.

Previously, it has been shown that sialidases, which remove sialyl residues from cells, are required for cells to adhere to the endothelial surface and to squeeze through the interendothelial junction. In addition, it has also been shown that inhibition of sialidases, either by antibody or sialidase inhibitors, reduced the recruitment of polymorphonuclear leukocytes to inflammatory sites. Currently no other therapies target sialic acid modification on eukaryotic cells as a method to treat inflammation. Many other therapies have sought to inhibit the function of specific molecules on the surface of cells involved in the ability of polymorphonuclear leukocytes to migrate to inflammatory sites; however, these target only one of many molecules involved in the process of diapedesis, and to date have not been very successful. In contrast, modification of cell surface sialic acid content is involved at multiple steps during diapedesis, and therefore may be more effective at inhibiting inflammation (3-6).

Also, overexpression of ST activity has been associated with oncogenic transformation and increased metastatic potential. Sialyltransferases also play a pivotal role in the maturation and function of myeloid cells and B and T lymphocytes. For example, CD22, a member of the immunoglobulin superfamily of adhesion molecules, is a B cell-restricted transmembrane protein required for lymphocyte activation and immune function. Sialyltransferase-mediated sialylation of CD22 abrogates CD22-mediated B and T lymphocyte adhesion essential to lymphocyte function. In the case of CD8+ T lymphocytes, ST activity dictates whether a cell undergoes apoptosis or progresses to become a viable memory T cell.

There is a recognized need in the art for the treatment of diseases or disorders characterized by excessive or unwanted inflammation. In addition, there is a recognized need in the art for reducing or preventing the metastatic potential of a tumor. Thus, the prior art is deficient in methods of inhibiting the activity of sialyltransferases. More specifically, the prior art is deficient in methods of inhibiting the activity of sialytransferases to reduce the ability of polymorphonuclear leukocytes to migrate across endothelial cells. The present invention fulfills this long-standing need and desire in the art.

### SUMMARY OF THE INVENTION

The present invention is directed to a method of reducing a level of activity of a sialyl transferase enzyme in a cell. The method comprises contacting a cell exhibiting a sialyl transferase activity with an amount of an inhibitor effective to inhibit the activity.

The present invention also is directed to a method of treating a pathophysiological condition in a subject. The method comprises administering to the subject a pharmacologically effective amount of an inhibitor of an activity of a sialyl transferase enzyme. The present invention is also directed to a related method comprising a further step of administering to the subject a pharmacologically effective amount of an inhibitor of an activity of sialidase enzyme.

The present invention is directed further to a related method of treating a pathophysiological condition in a subject. The related method comprises administering to the subject pharmacologically effective amounts of an inhibitor of an activity of a sialyl transferase enzyme and an inhibitor of an activity of sialidase enzyme.

The present invention is directed further yet to a method for screening for a potential inhibitor of a sialyl transferase enzyme. The method comprises selecting a potential sialyl transferase inhibitor and contacting cells effective to accept transfer of a sialyl residue from a fluorescently labeled substrate molecule with the inhibitor. The level of fluorescence in the presence or absence of the potential inhibitor is measured and the level of fluorescence in the presence of the potential inhibitor is compared with the level of fluorescence in the absence of the potential inhibitor. A decrease in fluorescence in the presence of the inhibitor is indicative that the inhibitor has an ability to inhibit sialyl transferase activity.

The present invention is directed further still to an inhibitor of a sialyl transferase enzyme screened by the method described herein.

Other and further aspects, features, benefits, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention are briefly summarized. The above may be better understood by reference to certain embodiments thereof which are illustrated in the appended drawings. These drawings form a part of the specification. It is to be noted; however, that the appended drawings illustrate preferred embodiments of the invention and therefore are not to be considered limiting in their scope.

FIGS. 1A-1C depict a sialyltransferase assay using sheep erythrocytes as sialyl residue acceptors. A complete reaction mixture consisting of neuraminidase-treated SRBCs, purified α2,6 sialyltransferase, and cytidine monophosphate (CMP)-5′ fluoresceinyl-N-acetylneuraminic acid (CMP-NANA) gave a strong signal (black) (FIGS. 1A-1C, MFI=637). Omission of the CMP-NANA (grey, FIG. 1A, MFI=72), or sialyltransferase (grey, FIG. 1B, MFI=219) or heat inactivation (100° C. for 30 min) of sialyltransferase (grey, FIG. 1C, MFI=205) reduced the fluorescence of the SRBCs. No signal was evident when the SRBC acceptor was omitted.

FIGS. 2A-2D depict human polymorphonuclear leukocytes as source of endogenous, i.e. CMP-inhibitable, sialyltransferase activity: While pretreatment of polymorphonuclear leukocytes with neuraminidase and exogenous sialyltransferase provided maximal sialyltransferase activity (FIG. 2A, MFI=406 v. 165 with CMP), a strong signal was evident when exogenous sialyltransferase was added to polymorphonuclear leukocytes unexposed to neuraminidase pretreatment (FIG. 2B, MFI=310 v. 167 with CMP). When polymorphonuclear leukocytes were pretreated with neuraminidase and intact, untreated polymorphonuclear leukocytes were added as an endogenous source of sialyltransferase, readily detectable sialyltransferase activity was evident (FIG. 2C, MFI=295 v. 141 with CMP). When an aliquot of intact, untreated polymorphonuclear leukocytes were added to polymorphonuclear leukocytes that were not exposed to neuraminidase, a lesser, but distinct signal was apparent (FIG. 2D, MFI=266 v. 131 with CMP). Transferase activity (solid bar) was compared in each case to polymorphonuclear leukocytes in which no exogenous sialyltransferase was added and in the presence of the specific sialyltransferase inhibitor, CMP (0.3 mM final concentration) (open bar).

FIGS. 3A-3B demonstrate that polymorphonuclear leukocytes monophosphate inhibits endogenous sialyltransferase activity. In FIG. 3A polymorphonuclear leukocytes (8×10⁶) were added to 10 μM CMP-NANA in the absence (solid peak) or presence (open peak) of 0.3 mM CMP and analyzed by flow cytometry. CMP treatment reduced the mean fluorescence intensity (MFI) from 364 to 222. In FIG. 3B to determine if intact, untreated polymorphonuclear leukocytes exhibit some basal level of sialidase activity necessary for ST activity, polymorphonuclear leukocytes were analyzed in the absence (solid peak) or presence (open peak) of 2-DN, an inhibitor of sialidase activity. A small, but highly reproducible reduction in ST activity was noted (reduction in MFI from 364 to 317). The data show a single experiment that was repeated independently 3 times.

FIG. 4 depicts inhibition of transendothelial cell migration by an ST inhibitor, cytidine monophosphate (CMP). Calcein AM-labeled human polymorphonuclear leukocytes were added to human pulmonary artery endothelial cells (HPAEC) pre-treated for 4 hrs with rTNF (50 U/ml). TEM was measured in response to IL-8 (150 ng/ml) over 2 hr as previously described (6) in the absence and presence of 0.3 mM CMP. 4 chambers/variable were studied. This figure is representative of 3 independent experiments.

FIG. 5 demonstrates that CMP inhibits the initial binding of non-activated polymorphonuclear leukocytes to the human microvascular endothelial cells (HMVEC). This figure is representative of the average of 2 independent experiments (N=10).

### DETAILED DESCRIPTION OF THE INVENTION

In one embodiment of the present invention there is provided a method of reducing a level of activity of a sialyl transferase enzyme in a cell, comprising contacting a cell exhibiting a sialyl transferase activity with an amount of an inhibitor effective to inhibit the activity.

In this embodiment the inhibitor may be cytidine monophosphate or an analog thereof or an antibody directed against said sialyl transferase enzyme. In one aspect of this embodiment the level of activity of sialyl transferase is associated with an inflammatory disease or disorder in a subject. Examples of an inflammatory disease or disorder are arthritis, inflammatory bowel disease or pulmonary inflammation. In another aspect the level of activity of sialyl transferase is associated with a primary cancer or the metastatic potential of a primary cancer.

In another embodiment of the present invention there is provided a method of treating a pathophysiological condition in a subject, comprising administering to the subject a pharmacologically effective amount of an inhibitor of an activity of a sialyl transferase enzyme.

Further to this embodiment the method may comprise administering to the subject a pharmacologically effective amount of an inhibitor of an activity of sialidase enzyme. Examples of sialidase inhibitors are 2,3 deoxy-N-acetyl neuraminic acid or an antibody directed against said sialidase enzyme. In this further embodiment the sialidase inhibitor may be administered prior to administration of the sialyl transferase inhibitor, concurrent with administration of the sialyl transferase inhibitor or subsequent to administration of the sialyl transferase inhibitor. Also, the sialidase activity may be associated with recruitment of polymorphonuclear cells to sites of inflammation in the subject. Furthermore, in both embodiments the sialyl transferase activity may be associated with migration of polymorphonuclear cells across endothelial cells.

In both embodiments the inhibitor may be cytidine monophosphate. Alternatively, the inhibitor may be an antibody (monoclonal or polyclonal) directed against a sialyl transferase enzyme. In one aspect of these embodiments the pathophysiological condition is an inflammatory disease or disorder. Representative examples are arthritis, inflammatory bowel disease or pulmonary inflammation. In another aspect the pathophysiological condition is a primary cancer or metastatic cancer.

In a related embodiment the present invention provides a method of treating a pathophysiological condition in a subject, comprising administering to the subject pharmacologically effective amounts of an inhibitor of an activity of a sialyl transferase enzyme and an inhibitor of an activity of sialidase enzyme. The sialidase inhibitor may be administered as described supra.

In this related embodiment the sialyl transferase activity and the sialidase activity are associated with migration of polymorphonuclear cells across endothelial cells and the recruitment thereof to sites of inflammation in the subject. Also, in this embodiment the sialyl transferase inhibitor may be cytidine monophosphate or an analog thereof or an antibody directed against directed against the sialyl transferase enzyme. In addition, the sialidase inhibitor may be 2,3 deoxy-N-acetyl neuraminic acid or an antibody directed against said sialidase enzyme. Furthermore, the pathophysiological conditions may be as described supra.

In yet another embodiment of the present invention there is provided a method for screening for a potential inhibitor of a sialyl transferase enzyme, comprising selecting a potential sialyl transferase inhibitor; contacting cells effective to accept transfer of a sialyl residue from a fluorescently labeled substrate molecule with the inhibitor; measuring the level of fluorescence in the presence or absence of the potential inhibitor; and comparing the level of fluorescence in the presence of the potential inhibitor with the level of fluorescence in the absence of the potential inhibitor, wherein a decrease in fluorescence in the presence of the inhibitor is indicative that the inhibitor has an ability to inhibit sialyl transferase activity.

In this embodiment the potential inhibitor potential inhibitor may be an antibody directed against a sialyl transferase enzyme or an inhibitory compound. An example of an inhibitory compound is a derivative or analog of cytidine monophospate. In one aspect of this embodiment the screened inhibitor is effective to reduce inflammation associated with an inflammatory disease or disorder. Examples of inflammatory disease or disorder are arthritis, inflammatory bowel disease and pulmonary inflammation. In another aspect the screened inhibitor is effective to treat a cancer or to reduce or eliminate the metastatic potential of a cancer.

In a related embodiment there is provided an inhibitor of a sialyl transferase enzyme screened by the method described supra. In this related embodiment a pharmaceutical composition may comprise the inhibitor and a a pharmaceutically acceptable carrier.

As used herein, the term, “a” or “an” may mean one or more. As used herein in the claim(s), when used in conjunction with the word “comprising”, the words “a” or “an” may mean one or more than one. As used herein “another” or “other” may mean at least a second or more of the same or different claim element or components thereof.

As used herein, the term “contacting” refers to any suitable method of bringing an inhibitory agent into contact with a sialyl transferase or sialidase protein or polypeptide or fragment thereof or a cell comprising the same. In vitro or ex vivo this is achieved by exposing the comprising the same to the inhibitory agent in a suitable medium. For in vivo applications, any known method of administration is suitable as described herein.

As used herein, the terms “treating” or “treatment” includes prophylactic treatment as well as alleviation of ongoing or intermittent symptoms occurring in an inflammatory disease or disorder or in a disease or disorder with an inflammatory component or in a primary cancer or metastatic cancer.

As used herein, the term “sialyl transferase inhibitor” means a molecular entity of natural, semi-synthetic or synthetic origin that blocks, stops, inhibits, and/or suppresses an activity of sialyl transferase enzyme, particularly the ability of polymorphonuclear cells to migrate across endothelial cells.

As used herein, the term “sialidase inhibitor” means a molecular entity of natural, semi-synthetic or synthetic origin that blocks, stops, inhibits, and/or suppresses an activity of sialidase enzyme, particularly the recruitment of polymorphonuclear cells to inflammatory sites. Inhibitors of sialidase are disclosed in U.S. Pat. Nos. 5,631,283 and 6,066,323 to the inventors, the contents of which is hereby incorporated by reference.

As used herein, the term “subject” refers to any target of the treatment.

Provided herein is a method of treating inflammation. STs must restore sialyl residues to the polymorphonuclear leukocyte surface so that these cells can “de-adhere” from the abluminal surface of the endo-thelial cells. STs restore sialyl residues to the polymorphonuclear leukocyte surface. Inhibition of ST by cytidine monophosphate reduces the ability of polymorphonuclear leukocytes to migrate across endothelial cells. Thus, inhibition of either the sialidase or sialyl transferase can reduce migration and consequently inflammation. Moreover, a combination of inhibitors of sialidase and ST will be even more effective. Since STs are essential to the ability of many tumor cells to metastasize, ST inhibitors are also useful for the therapeutic treatment of cancer.

Activated polymorphonuclear leukocytes mobilize endogenous sialidase activity from an intracellular compartment to the plasma membrane. Removal of as little as 20% of total cell-associated sialic acid residues profoundly increase polymorphonuclear leukocyte adherence to and migration across endothelial cell monolayers. Inhibition of endogenous sialidase activity in vitro prevented these PMN-endothelial cell interactions and in vivo reduced polymorphonuclear leukocyte recruitment to inflammatory sites. Moreover, fMLP-activated polymorphonuclear leukocytes directly desialylate the surface of HPAECs, which in turn increases polymorphonuclear leukocyte migration across the EC monolayer.

Since polymorphonuclear leukocytes must dynamically respond to a range of dissimilar environmental stimuli, these cells require the capacity to restore sialyl residues to surface glycoconjugates. It is demonstrated herein that intact human polymorphonuclear leukocytes also have ST activity that catalyzes the transfer of sialyl residues onto host cells, including polymorphonuclear leukocytes. Thus, polymorphonuclear leukocytes have the capacity to rapidly alter their (and/or other) cell surface(s) either through the removal (sialidase) or restoration (ST) of sialyl residues. It is this modulation which regulates PMN adhesion and motility.

Although it has been inferred that polymorphonuclear leukocytes have ST activity within the Golgi complex that enables polymorphonuclear leukocytes to restore sialyl residues during the recycling of surface receptors, there have been no reports of ST activity on the surface of polymorphonuclear leukocytes. It is demonstrated herein that the ST activity of polymorphonuclear leukocytes is localized to the cell surface, and found that intact, viable polymorphonuclear leukocytes displayed ST activity. As a result it can be inferred polymorphonuclear leukocytes migrate through the endothelial paracellular pathway without disrupting barrier integrity, and both sialidase and ST activities might be simultaneously expressed but spatially segregated within distinct subcellular compartments in polarized cells.

It is contemplated that sialidase activity concentrated on the leading front of a migrating polymorphonuclear leukocyte may remove sialyl residues from glycoconjugates at the inter-endothelial cell junction, such as CD31, thereby facilitating their adherence to as well as their ability to squeeze through this junction, whereas STs at the polymorphonuclear leukocyte “tail” may restore these residues to the cell surface allowing for physical disengagement from substrates enabling polymorphonuclear leukocytes to continue migration through tissues. This shows how polymorphonuclear leukocytes can negotiate the paracellular pathway without opening the pathway to macromolecules or fluid. It is also contemplated that polymorphonuclear leukocyte ST activity can resialylate endothelial cell surfaces thereby “resealing” the paracellular pathway.

In addition the sialidase/ST enzyme system, through its dynamic control of polymorphonuclear leukocyte surface sialylation, regulates the cell-cell and likely the cell-matrix interactions that are prerequisites to PMN motility. The constitutive ST activity of intact, untreated polymorphonuclear leukocytes demonstrated herein insures that polymorphonuclear leukocytes do not become promiscuously adherent in response to every transient, minimal stimulus. Furthermore, the coordinated activities of sialidase and ST activities in polymorphonuclear leukocyte allow a rapidly reversible, highly localized response to an inflammatory stimulus and can serve as a therapeutic target.

Thus, the present invention provides a method of inhibiting an activity of a sialyl transferase using an inhibitory compound to inhibit the migration of polymorphonuclear cells across endothelial cells. Preferably, such inhibitor is, although not limited to, cytidine monophosphate (CMP) or an analog thereof. Alternatively, an inhibitor may be an antibody directed against a sialyl transferase. For example, production and identification of monoclonal antibodies are utilize standard molecular biological techniques well known in the art. Such inhibitors may be formulated as pharmaceutical compositions comprising a pharmaceutically acceptable carrier as is known and standard in the art.

It is contemplated that potential inhibitors of sialyl transferases may be screened using suitable assays known and standard in the art. A suitable inhibitor would inhibit the transfer of sialyl residues to an appropriate acceptor, such as polymorphonuclear cells. For example, a simple decrease in fluorescent levels in the presence of the potential inhibitor is indicative the potential inhibitor is effective to inhibit enzyme activity. For example, potential inhibitors may be inhibitory compounds designed based on the structure of cytidine monophosphate. Also, potential inhibitory compounds may be designed based on the structure of the human sialyl transferase enzymes or homologs thereof using in part computer aided design as is known in the art. Alternatively, a potential inhibitor may be an antibody directed against a sialyl transferase enzyme.

Inhibitors of the sialyl transferase enzymes are useful to prevent or to treat a pathophysiological condition in a subject such as inflammatory diseases and disorders or diseases and disorders having an inflammatory component. For example, inhibitors of sialyl transferase enzymes, upon contacting such enzyme or cells comprising the enzyme or cells whose activities are regulated by an activity of the enzyme, may demonstrate a therapeutic effect against arthritis, inflammatory bowel disease or pulmonary inflammation. Alternatively, inhibitors of sialyl transferase enzymes may demonstrate a chemotherapeutic effect against a cancer, including reducing or preventing metastasis of the cancer.

It is also contemplated that inhibition of sialidase enzyme increases the therapeutic benefits obtained from inhibition of a sialyl transferase enzyme. An inhibitor of sialidase is useful to inhibit, reduce or prevent the recruitment of polymorphonuclear cells to inflammatory sites. The sialidase inhibitor may be brought into contact with sialidase enzyme, or cells comprising the enzyme or cells whose activities are regulated by an activity of the enzyme prior to, concurrently or subsequently to contacting a sialyl transferase enzyme with an inhibitor thereof. Inhibitors of sialidase may be an antibody or other inhibitory compound, for example, but not limited to, 2,3 deoxy-N-acetyl neuraminic acid, as are known in the art and disclosed in U.S. Pat. Nos. 5,631,283 and 6,066,323.

Dosage formulations of inhibitors of a sialyl transferase and sialidase may comprise conventional non-toxic, physiologically or pharmaceutically acceptable carriers or vehicles suitable for the method of administration. These compounds or pharmaceutical compositions thereof may be administered independently one or more times to achieve, maintain or improve upon a pharmacologic or therapeutic effect derived from these compounds or other agents suitable for the disease or disorder being treated. It is well within the skill of an artisan to determine dosage or whether a suitable dosage comprises a single administered dose or multiple administered doses. An appropriate dosage depends on the subject's health, the progression or remission or at risk status of the disease or disorder, the route of administration and the formulation used.

The following examples are given for the purpose of illustrating various embodiments of the invention and are not meant to limit the present invention in any fashion.

### EXAMPLE 1

Materials and Methods

Cell Preparation

Human red blood cells (RBCs) were obtained from the peripheral blood of healthy volunteers by venipuncture under a protocol approved by the Institutional Review Board (IRB). Sterile and defibrinated sheep red blood cells (SRBC) were purchased from Waltz Farm (Smithsburg, Md.) and stored at 4° C. polymorphonuclear leukocytes were processed from the peripheral blood of healthy human donors as previously described (3) under the same IRB-approved protocol.

Reagents

Type-5 neuraminidase (Clostridium perfringens), 2,3 deoxy-N-acetyl neuraminic acid (2-DN) and cytidine 5′-monophosphate (CMP) were purchased from Sigma (St. Louis, Mo.). CMP-5-fluoresceinyl-neuraminic acid (CMP-5-NANA) was purchased from (Calbiochem, San Diego, Calif.). Alpha 2,6 sialyltransferase (a 2,6 ST) was purchased from Genzyme (Cambridge, Mass.) and protease inhibitor cocktail tablets were purchased from Roche (Indianapolis, Ind.).

Sialyltransferase Assay

For the study of PMN ST activity, a previously described ST assay was adapted in which CMP-5-NANA served as the labeled sialyl residue donor and neuraminidase-treated RBCs as the sialyl residue acceptor (7). Based on preliminary studies, a standard reaction mixture contained a sialyl residue acceptor, either RBCs or polymorphonuclear leukocytes, CMP-5-NANA and a ST source. Purified α2,6-ST at a final concentration of 0.5 nU/ml, or polymorphonuclear leukocytes as an endogenous source of ST activity were used in a final volume of 100 μl cell incubation buffer (CIB) containing PBS plus 5 mg/ml BSA, pH 6.8. Sialyl residue acceptor SRBCs and polymorphonuclear leukocytes were desialylated with 25 mU/ml NANase/1-2×10⁸/cells in 1 ml of CIB for 1 h at 37° C., washed once in CIB and used at 4×10⁶ cells or at 8×10⁶ cells respectively in a volume of 40 μl. When used as an endogenous source of ST activity, human polymorphonuclear leukocytes were adjusted to a stock concentration of 1×10⁸ PMN/ml, lysed by freeze/thawing, centrifuged, and the protein concentration of the supernatant determined (Bio-Rad Laboratories, Hercules, Calif.). Intact human polymorphonuclear leukocytes at 8×10⁶/0.1 ml assay volume were also used both as a source of ST activity as well as an acceptor of sialyl residues.

After gentle mixing, all tubes were incubated for 2 h at 37° C. in a humidified atmosphere containing 5% CO₂. The cells were washed, pelleted and resuspended in CIB and analyzed fluorocytometrically on a FACScan II (Becton-Dickinson, Mountain View, Calif.). Based on forward and right angle light scatter properties, an electronic gate was placed around the RBCs or polymorphonuclear leukocytes which were excited at 488 nM and emission measured at 530 nM. At least 10,000 events were recorded per condition. Data was analyzed with a CellQuest analysis program.

For each experiment, controls included omission of each of the components of the assay mixture, i.e., ST source, CMP-5 NANA or sialyl residue acceptor) as well as boiled, exogenous α2,6 ST. When indicated, 2-DN (0.04 mM, final concentration), an inhibitor of sialidase activity (8) was introduced to prevent the possible removal of the transferred sialyl residues. ST activity was measured after subtraction of background fluorescence, i.e., complete assay mixture minus either the exogenous ST or, for endogenous ST, in the presence and absence of the ST inhibitor, CMP (0.3 mM final concentration) and was expressed as % of maximal activity. For experiments in which it was estimated the endogenous ST activity, a standard curve was constructed using serial 2-fold dilutions of purified α2-6 ST enzyme from which tissue ST activity was interpolated, and expressed the ST activity as nU per μg protein.

Transendothelial Migration of PMNs

The ability of human polymorphonuclear leukocytes to migrate across human pulmonary artery endothelial cell (HPAEC) monolayers in response to a chemotactic stimulus (IL-8) was measured as previously described (6). Only EC monolayers retaining <97% of a ¹⁴C-bovine serum albumen tracer were studied (i.e. no loss of barrier function). After treatment of HPAECs for 4 h with 25 U/ml TNFr, calcein AM (Molecular Probes, Eugene, Oreg.)-labeled human polymorphonuclear leukocytes (5×10⁵ cells/well) were introduced into the upper compartments of the assay chambers, incubated for 2 h at 37° C., after which time each lower compartment was sampled and fluorometrically assayed. A standard curve was established for each experiment from which polymorphonuclear leukocyte numbers could be interpolated from fluorescence units and PMN TEM was expressed as % migration.

### EXAMPLE 2

Exogenous ST Transfers Sialyl Residues onto Sheep RBCs

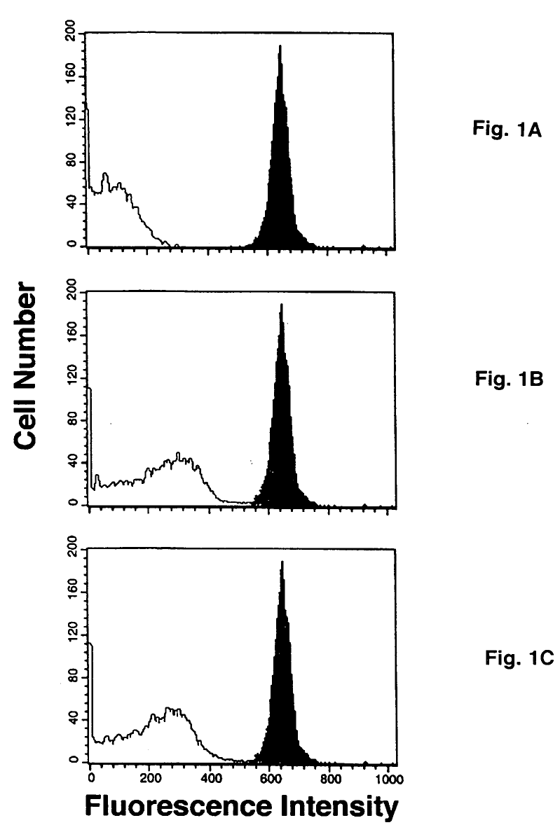

In preliminary experiments it was established that neuraminidase treatment prepared the surface of both human and sheep RBCs for the comparable acceptance of ST-mediated transfer of CMP-5-NANA (data not shown). The complete mixture of sheep red blood cells, CMP-5-NANA and exogenous ST resulted in a strong signal on flow cytometry (FIGS. 1A-1C, dark peaks). Omission of either CMP-5-NANA, i.e., autofluorescence, (FIG. 1A, grey peak) or exogenous ST (FIG. 1B, grey peak) or use of heat-inactivated ST (FIG. 1C, grey peak) from the reaction mixture markedly reduced the signal. In the absence of neuraminidase pretreatment, sheep red blood cells displayed no fluorescent signal, i.e., sheep red blood cells were unable to act as an acceptor for the labeled CMP-NANA.

### EXAMPLE 3

Intact PMNs Both as ST Source and as Sialyl Residue Acceptors

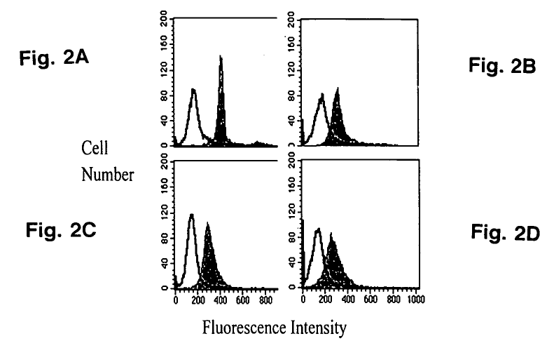

Since RBCs were capable of accepting sialyl residues, it was contemplated whether intact human polymorphonuclear leukocytes might also function as sialyl residue acceptors in the presence of exogenous (bacterial) ST activity. Exogenous ST mediated the transfer of CMP-5-NANA onto polymorphonuclear leukocytes that had been pretreated with neuraminidase (FIG. 2A, solid peak). There was an increase in the ability of polymorphonuclear leukocytes to accept sialyl residues with increasing doses of neuraminidase pretreatment until a plateau was attained at 30 mU neuraminidase/ml (data not shown). This ST activity was inhibited by CMP (open peak). Exogenous ST also mediated CMP-5-NANA transfer to polymorphonuclear leukocytes without prior neuraminidase treatment, but here the effect was diminished (FIG. 2B). Thus, even in the absence of neuraminidase pretreatment, sialyl residue acceptor sites were available on intact human polymorphonuclear leukocytes.

In assaying endogenous sources for ST activity, a specific ST inhibitor (9, 10), CMP, is utilized to verify that the assay system was in fact detecting ST activity. Since preliminary tests demonstrated that 0.3 mM CMP fully inhibited ST activity in vitro and was not toxic to the polymorphonuclear leukocytes, each assay for endogenous ST activity was performed in the presence and absence of CMP. The difference in corrected values generated in the presence and absence of CMP was taken as the true ST activity, i.e., CMP-inhibitable activity.

Since polymorphonuclear leukocyte lysates contained ST activity, it was contemplated whether intact polymorphonuclear leukocytes also could serve as an endogenous source for ST. To optimally expose more sialyl acceptor sites, an aliquot of polymorphonuclear leukocytes was pretreated with neuraminidase and added to the reaction mixture as a sialyl residue acceptor, and an aliquot of untreated polymorphonuclear leukocytes was added in place of the purified enzyme as a source of ST activity (FIG. 2C). Under these conditions CMP-inhibitable ST activity was evident indicating that intact polymorphonuclear leukocytes express ST activity that was presentable to neighboring polymorphonuclear leukocytes that had been “prepared” with exogenous neuraminidase.

Since polymorphonuclear leukocytes can serve as acceptor cells in the ST assay in the absence of neuraminidase pretreatment (FIG. 2B), it was contemplated whether intact polymorphonuclear leukocytes that were not pretreated with neuraminidase could be used as both the sialyl residue acceptor and endogenous source of ST activity. After incubation with CMP-5-NANA, CMP-inhibitable ST activity was evident in intact, untreated cells (FIG. 2D). These data suggest that during an inflammatory response, intact polymorphonuclear leukocytes can mediate the autocrine/paracrine transfer of sialyl residues from polymorphonuclear leukocyte to polymorphonuclear leukocyte.

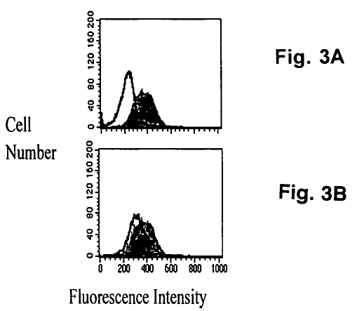

Since RBCs not treated with neuraminidase cannot serve as sialyl residue acceptors, it was contemplated that for polymorphonuclear leukocytes to serve as sialyl residue acceptors in the absence of prior neuraminidase treatment, some previously unrecognized constitutive level of endogenous sialidase activity may expose acceptor sites on the PMN surface to accept sialyl residues. To test this hypothesis, unstimulated, intact polymorphonuclear leukocytes that were not exposed to neuraminidase had CMP-inhibitable activity (FIG. 3A). The addition of CMP to the ST assay mixture reduced the mean fluorescence intensity (MFI) from 364 to 222. Addition of the sialidase inhibitor, 2-DN, to those same untreated polymorphonuclear leukocytes resulted in a small, but consistently reproducible decrement in ST activity (decrease in MFI from 364 to 317) (FIG. 3B). Therefore, in non-stimulated polymorphonuclear leukocytes, a basal level of sialidase activity appears necessary for the constitutive turnover of surface sialyl residues, perhaps as part of cell surface remodeling. This activity was not detectable with the less sensitive biochemical assays of sialidase activity used in our earlier reports (3-6) and was insufficient to cause polymorphonuclear leukocyte functional changes normally associated with desialylation.

### EXAMPLE 4

Transendothelial Migration

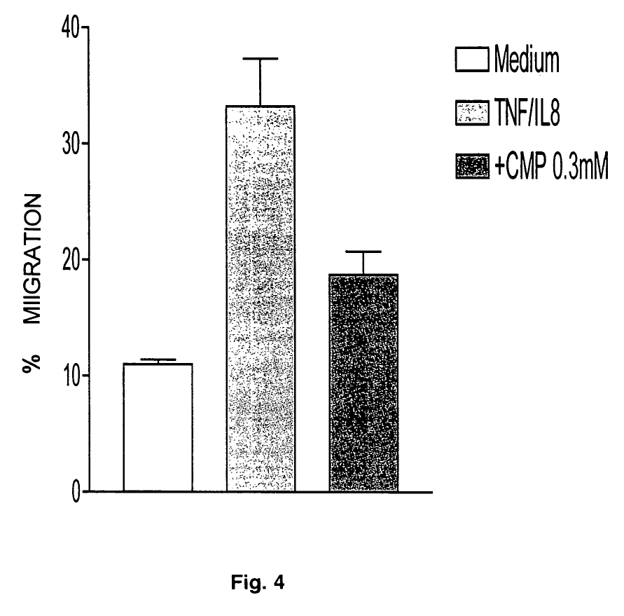

It is contemplated that once migrating PMNs reached the abluminal surface of endothelial cells, restoration of sialyl residues to the PMN surface would be required for them to “de-adhere” and to continue their migration. There was a robust (>3-fold increase) migration of calcein-AM labeled human polymorphonuclear leukocytes across the TNFα-treated HPAEC monolayers in response to IL-8 (FIG. 4). Addition of the ST inhibitor, CMP, inhibited TEM >40% (p<0.03).

### EXAMPLE 5

Adherence of Resting PMNs to Endothelial Cells

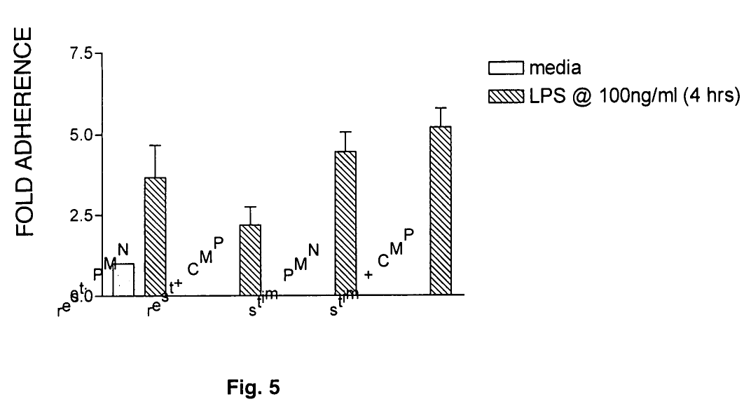

Non-stimulated or resting polymorphonuclear leukocytes added to media treated endothelial cells demonstrate little adherence. In contrast, when the human microvascular endothelial cells, HMVECs, are treated with lipopolysaccharide, LPS, (100 ng/ml for 4 hrs) to activate them, resting polymorphonuclear leukocytes adhere better to the HMVECs. However, in the presence of the inhibitor, CMP, (2.5 mM for 5 minutes prior to stimulation) there is decreased adherence. When polymorphonuclear leukocytes are activated with the tetrapeptide FMLP (1×10⁻⁷ M/cyt b for 5 min.) they adhere better than do resting polymorphonuclear leukocytes (FIG. 5). At this later stage of PMN-EC interaction, sialyltransferase doesn't play a role. It is contemplated that CMP appears to work by inhibiting the initial binding of non-activated polymorphonuclear leukocytes to the ECs and also works at the late stage where polymorphonuclear leukocytes that have migrated through the EC monolayer are unable to move further to the inflammatory nidus.

### EXAMPLE 6

In Vivo Effects of Cytidine Monophosphate on Binding of PMNs

Mice are pre-treated intravenously with different doses of CMP 30 minutes before giving IL-8 (150 ng/ml) intraperitoneally to recruit polymorphonuclear leukocytes. This is followed with another dose of CMP one hr after the IL-8. Polymorphonuclear leukocytes in the peritoneal fluid of these mice are counted at 2 hrs after the IL-8, i.e., one hr after the second dose of CMP. The total polymorphonuclear leukocytes in the perioneal fluid of mice treated with CMP is compared with those given an equal volume of saline. It is contemplated that those receiving the CMP will have a lower number of polymorphonuclear leukocytes in the perioneal fluid.

The following references are cited herein.
1. Muller W A. 2002. Leukocyte-endothelial cell interactions in the inflammatory response. Lab. Investig. 82:521.
2. Pilatte et al. 1993. Sialic acids as important molecules in the regulation of the immune system: pathophysiological implications of sialidases in immunity. Glycobiol 3:201.
3. Cross A. S., and G. Wright. 1991. The mobilization of sialidase from intracellular stores to the surface of human neutrophils and its role in stimulated adhesion responses of these cells. J Clin Invest. 88:2067-76.
4. Stamatos et al. 1997. Desialylation of peripheral blood mononuclear cells promotes growth of HIV-1. Virology 228:123.
5. Cross et al. 2003. Recrutiment of murine neutrophils in vivo through endogenous sialidase activity. J. Biol. Chem 278:4112.
6. Sakarya et al. 2004. Mobilization of neutrophil sialidase activity desialylates the pulmonary vascular endothelial surface and increases resting neutrophl adhesion to and migration across the endothelium. Glycobiol. 14:481.
7. Sticher, U. and R. Brossmer. 1990. Highly sensitive fluorometric assay for transferase activity using fluoresceinyl-NeuAc as donor. Analyt. Biochem. 186:127.
8. Mendla K., and M. Cantz. 1984. Specificity studies on the oligosaccharide neuraminidase of human fibroblasts. Biochem. J. 218:625.

Any publications or patents mentioned in this specification are indicative of the levels of those skilled in the art to which the invention pertains. Further, these publications are incorporated by reference herein to the same extent as if each individual publication was specifically and individually incorporated by reference.

One skilled in the art will readily appreciate that the present invention is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A method of reducing a level of activity of a sialyl transferase enzyme in a cell, comprising:
contacting a cell exhibiting a sialyl transferase activity with an amount of an inhibitor effective to inhibit said activity.

2. The method of claim 1, wherein said inhibitor is cytidine monophosphate or an analog thereof or an antibody directed against said sialyl transferase enzyme.

3. The method of claim 1, wherein said level of activity of sialyl transferase is associated with an inflammatory disease or disorder in a subject.

4. The method of claim 3, wherein said inflammatory disease or disorder is arthritis, inflammatory bowel disease or pulmonary inflammation.

5. The method of claim 1, wherein said level of activity of sialyl transferase is associated with a primary cancer or the metastatic potential of a primary cancer.

6. A method of treating a pathophysiological condition in a subject, comprising:
administering to the subject a pharmacologically effective amount of an inhibitor of an activity of a sialyl transferase enzyme.

7. The method of claim 6, further comprising administering to the subject a pharmacologically effective amount of an inhibitor of an activity of sialidase enzyme.

8. The method of claim 7, wherein said sialidase inhibitor is 2,3 deoxy-N-acetyl neuraminic acid or an antibody directed against the sialidase enzyme.

9. The method of claim 7, wherein said sialidase inhibitor is administered prior to administration of said sialyl transferase inhibitor, concurrent with administration of said sialyl transferase inhibitor or subsequent to administration of said sialyl transferase inhibitor.

10. The method of claim 7, wherein said sialidase activity is associated with recruitment of polymorphonuclear cells to sites of inflammation in the subject.

11. The method of claim 6, wherein said sialyl transferase activity is associated with migration of polymorphonuclear cells across endothelial cells.

12. The method of claim 6, wherein said inhibitor is cytidine monophosphate or an analog thereof or an antibody directed against said sialyl transferase enzyme.

13. The method of claim 6, wherein the pathophysiological condition is an inflammatory disease or disorder.

14. The method of claim 13, wherein said inflammatory disease or disorder is arthritis, inflammatory bowel disease or pulmonary inflammation.

15. The method of claim 67, wherein the pathophysiological condition is a primary cancer or metastatic cancer.

16. A method of treating a pathophysiological condition in a subject, comprising:
administering to the subject pharmacologically effective amounts of an inhibitor of an activity of a sialyl transferase enzyme and of an inhibitor of an activity of sialidase enzyme.

17. The method of claim 16, wherein said sialidase inhibitor is administered prior to administration of said sialyl transferase inhibitor, concurrent with administration of said sialyl transferase inhibitor or subsequent to administration of said sialyl transferase inhibitor.

18. The method of claim 16, wherein said sialyl transferase activity and said sialidase activity are associated with migration of polymorphonuclear cells across endothelial cells and the recruitment thereof to sites of inflammation in the subject.

19. The method of claim 16, wherein said sialyl transferase inhibitor is cytidine monophosphate or an analog thereof or an antibody directed against said sialyl transferase enzyme.

20. The method of claim 16, wherein said sialidase inhibitor is 2,3 deoxy-N-acetyl neuraminic acid or an antibody directed against said sialidase enzyme.

21. The method of claim 16, wherein the pathophysiological condition is an inflammatory disease or disorder.

22. The method of claim 21, wherein said inflammatory disease or disorder is arthritis, inflammatory bowel disease or pulmonary inflammation.

23. The method of claim 16, wherein the pathophysiological condition is a primary cancer or a metastatic cancer.

24. A method for screening for a potential inhibitor of a sialyl transferase enzyme, comprising:
selecting a potential sialyl transferase inhibitor;
contacting cells effective to accept transfer of a sialyl residue from a fluorescently labeled substrate molecule with said inhibitor;
measuring the level of fluorescence in the presence or absence of the potential inhibitor; and
comparing the level of fluorescence in the presence of the potential inhibitor with the level of fluorescence in the absence of the potential inhibitor, wherein a decrease in fluorescence in the presence of the inhibitor is indicative that the inhibitor has an ability to inhibit sialyl transferase activity.

25. The method of claim 24, wherein said potential inhibitor is an antibody directed against a sialyl transferase enzyme or an inhibitory compound.

26. The method of claim 27, wherein said inhibitory compound is a derivative or analog of cytidine monophospate.

27. The method of claim 24, wherein the screened inhibitor is effective to reduce inflammation associated with an inflammatory disease or disorder.

28. The method of claim 27, wherein said inflammatory disease or disorder is arthritis, inflammatory bowel disease or pulmonary inflammation.

29. The method of claim 24, wherein the screened inhibitor is effective to treat a cancer or to reduce or eliminate the metastatic potential of a cancer.

30. An inhibitor of a sialyl transferase enzyme screened by the method of claim 24.

31. A pharmaceutical composition comprising the inhibitor of claim 30 and a pharmaceutically acceptable carrier.
